(19) United States
(12) Patent Application Publication
(10) Pub. No.: US 2022/0112471 A1
(43) Pub. Date: Apr. 14, 2022

(54) NADH-DEPENDENT AMINO ACID DEHYDROGENASE AND APPLICATION THEREOF IN INCREASING LYSINE YIELD

(71) Applicant: TSINGHUA UNIVERSITY, Beijing (CN)

(72) Inventors: Zhen CHEN, Beijing (CN); Dehua LIU, Beijing (CN)

(73) Assignee: TSINGHUA UNIVERSITY, Beijing (CN)

(21) Appl. No.: 17/277,661

(22) PCT Filed: Aug. 20, 2019
(86) PCT No.: PCT/CN2019/101521

§ 371 Date: Mar. 18, 2021

(30) Foreign Application Priority Data
Sep. 18, 2018 (CN) 201811089319.0

(51) Int. Cl. C12N 9/06; C12N 9/02; C12P 13/08; C12P 13/00

(52) U.S. Cl. CPC C12N 9/0016; C12Y 104/01021; C12N 9/0008; C12P 13/001; C12N 9/0093; C12Y 104/01016; C12P 13/08; C12Y 102/01011

(57) ABSTRACT

Disclosed is an NADH-dependent amino acid dehydrogenase and an application thereof in increasing lysine yield. The amino acid dehydrogenases are aspartate dehydrogenase derived from Pseudomonas aeruginos, aspartate semialdehyde dehydrogenase derived from Tistrella mobilis, dihydropyridine dicarboxylic acid reductase derived from Mycobacterium tuberculosis, and diaminopimelate dehydrogenase derived from Tepidanaerobacter acetatoxydans. The amino acid sequences thereof are as shown in SEQ ID NOs: 1, 3, 5, and 7, respectively. NADH or both NADH and NADPH can be used as co-factors of the amino acid dehydrogenase to synthesize lysine, thereby reducing the demand for NADPH in the cell, and significantly increasing the production of lysine or pentanediamine.

### TECHNICAL FIELD

The present invention belongs to the field of gene engineering and biological fermentation technology, in particular to NADH-dependent amino acid dehydrogenase and application thereof.

### BACKGROUND

Lysine is an extremely important amino acid, and widely used in the field of feed additives, healthcare and medicines. At present, the industrial production of lysine is carried out mainly by a microbiological fermentation method. Strains commonly used in industry include Corynebacterium glutomicum and Escherichia coli. During biosynthesis of lysine, NADPH needs to be consumed for synthesizing lysine in a four-step enzymatic reaction process. Therefore, traditional methods balance coenzyme demands in a lysine synthesis process by enhancing the supply of NADPH, for instance, raising metabolic flux of the pentose phosphate pathway, or expressing NADPH-dependent glyceraldehyde-3-phosphate dehydrogenase, thereby increasing lysine yield. During the production of 1,5-pentanediamine, there also is a problem that NADPH is highly demanded, and 1,5-pentanediamine is obtained by expressing lysine decarboxylase in a lysine producing strain. In microorganism cells, the concentration of NADH is generally far greater than that of NADPH. NADH is more stable and cheaper than NADPH, and therefore there is great potential of utilizing NADH in cells to synthesize lysine or 1,5-pentanediamine.

### SUMMARY OF THE INVENTION

An objective of the present invention is to provide a NADH-dependent amino acid dehydrogenase and use thereof.

In the synthesis pathway of lysine and 1,5-pentanediamine, NADPH needs to be consumed in the following four steps: (1) aminating oxaloacetate to generate aspartic acid; (2) reducing aspartyl phosphate to generate aspartate semialdehyde; (3) reducing dihydrodipicolinic acid to generate piperidinedicarboxylate; and (4) reducing piperidinedicarboxylate to generate diaminopimelic acid. The majority of enzymes currently found in microorganisms for catalyzing the above steps strictly depended on NADPH. Through numerous bioinformatics analysis and repeated comparative experiments, by expressing genes of amino acid dehydrogenases from different sources in Escherichia coli, detecting catalytic activity of the enzymes and detecting coenzymes specificity on NADH and NADPH, it was found that an aspartate dehydrogenase (with amino acid sequence of SEQ ID NO: 1) derived from Pseudomonas aeruginos, an aspartate semialdehyde dehydrogenase (with amino acid sequence of SEQ ID NO: 3) derived from Tistrella mobilis, a dihydrodipicolinic acid reductase (with amino acid sequence of SEQ ID NO: 5) derived from Mycobacterium tuberculosis, and a diaminopimelic acid dehydrogenase (with amino acid sequence of SEQ ID NO: 7) derived from Tepidanaerobacter acetatoxydans can catalyze the above four steps by utilizing NADH.

Hence, the present invention provides a NADH-dependent amino acid dehydrogenase, comprising:

an aspartate dehydrogenase derived from Pseudomonas aeruginos;

an aspartate semialdehyde dehydrogenase derived from Tistrella mobilis;

a dihydrodipicolinic acid reductase derived from Mycobacterium tuberculosis; and/or

a diaminopimelic acid dehydrogenase derived from Tepidanaerobacter acetatoxydans;

wherein their amino acid sequences are set forth in SEQ ID NOs: 1, 3, 5 and 7, respectively, or are the amino acid sequences obtained by substitution, deletion and/or addition of one or more amino acids in the amino acid sequences set forth in SEQ ID NOs: 1, 3, 5 and 7, respectively, without affecting their bioactivities.

Through codon optimization, the present invention provides genes encoding the NADH-dependent amino acid dehydrogenases. The nucleotide sequences of the genes comprise the nucleotide sequences set forth in SEQ ID NOs: 2, 4, 6 and 8, respectively, or are the nucleotide sequences which have 90% or more homology with SEQ ID NOs: 2, 4, 6 and 8 obtained by substitution, deletion and/or addition of one or more bases as well as code amino acid dehydrogenases of the same function.

The present invention provides a biological material containing the genes. The biological material is a vector, a recombinant bacterium, a cell line or an expression cassette.

Preferably, the recombinant bacterium is a strain which can fermentatively produce lysine or 1,5-pentanediamine and contains or over-expresses the NADH-dependent amino acid dehydrogenase of the present invention.

The strain which can fermentatively produce lysine or 1,5-pentanediamine is Corynebacterium glutamicum or Escherichia coli.

More preferably, the recombinant bacterium is a strain which can fermentatively produce lysine or 1,5-pentanediamine and contains or over-expresses an aspartate dehydrogenase derived from Pseudomonas aeruginos and an aspartate semialdehyde dehydrogenase derived from Tistrella mobilis; or

contains an aspartate semialdehyde derived from Pseudomonas aeruginos, an aspartate semialdehyde dehydrogenase derived from Tistrella mobilis and a dihydrodipicolinic acid reductase derived from Mycobacterium tuberculosis; or

contains an aspartate dehydrogenase derived from Pseudomonas aeruginos, an aspartate semialdehyde dehydrogenase derived from Tistrella mobilis, a dihydrodipicolinic acid reductase derived from Mycobacterium tuberculosis and a diaminopimelic acid dehydrogenase derived from Tepidanaerobacter acetatoxydans.

Most preferably, the recombinant bacterium is a strain which can fermentatively produce lysine or 1,5-pentanediamine and in which a NADPH-dependent amino acid dehydrogenase is replaced with the NADH-dependent amino acid dehydrogenase of the present invention.

In one embodiment of the present invention, the recombinant bacterium AM3 is a mutant Corynebacterium glutamicum ATCC21543 in which the endogenous aspartate transaminase is replaced with a NADH-dependent aspartate dehydrogenase (with amino acid sequence of SEQ ID NO: 1), the endogenous NADPH-dependent aspartate semialdehyde transaminase is replaced with a NADH-dependent aspartate semialdehyde dehydrogenase (with amino acid sequence of SEQ ID NO: 3), and the endogenous NADPH-dependent dihydrodipicolinic acid reductase is replaced with a NADH-dependent dihydrodipicolinic acid reductase (with amino acid sequence of SEQ ID NO: 5), and the recombinant bacterium can remarkably increase lysine yield up to 27.7 g/L by fermentation in a fermentation medium.

In addition, the recombinant bacteria AM2 and AM4 in the examples of the present invention also have an excellent effect of increasing lysine yield, up to 25.8 g/L and 25.6 g/L, respectively. The recombinant bacterium AM2 is a Corynebacterium glutamicum ATCC21543 mutant in which the endogenous aspartate transaminase is replaced with a NADH-dependent aspartate dehydrogenase (with amino acid sequence of SEQ ID NO: 1), and the endogenous NADPH-dependent aspartate semialdehyde transaminase is replaced with a NADH-dependent aspartate semialdehyde dehydrogenase (with amino acid sequence of SEQ ID NO: 3).

The recombinant bacterium AM4 is a Corynebacterium glutamicum ATCC21543 mutant in which the endogenous aspartate transaminase is replaced with a NADH-dependent aspartate dehydrogenase (with amino acid sequence of SEQ ID NO: 1), the endogenous NADPH-dependent aspartate semialdehyde transaminase is replaced with a NADH-dependent aspartate semialdehyde dehydrogenase (with amino acid sequence of SEQ ID NO: 3), the endogenous NADPH-dependent dihydrodipicolinic acid reductase is replaced with a NADH-dependent dihydrodipicolinic acid reductase (with amino acid sequence of SEQ ID NO: 5), and the endogenous NADPH-dependent diaminopimelic acid dehydrogenase is replaced with a NADH-dependent diaminopimelic acid dehydrogenase.

The fermentation medium comprises (g/L): 80 g of glucose, 10 g of corn steep liquor, 4.5 g of urea, 45 g of ammonium sulfate, 0.5 g of potassium dihydrogen phosphate, 0.5 g of magnesium sulfate heptahydrate, 10 mg of ferrous sulfate heptahydrate, 10 mg of manganese sulfate tetrahydrate, 5 mg of β-alanine, 5 mg of nicotinic acid, 5 mg of thiamine hydrochloride, 0.3 mg of biotin, 30 g of calcium carbonate, 0.2 g of threonine, and 0.2 g of leucine, adding water to a volume of 1 L.

Further, the present invention provides use of the above-mentioned NADH-dependent amino acid dehydrogenases or coding genes thereof or a biological material containing the coding genes in the preparation of lysine or 1,5-pentanediamine.

The present invention provides use of the above-mentioned NADH-dependent amino acid dehydrogenase or coding genes thereof or a biological material containing the coding genes in increasing lysine yield or 1,5-pentanediamine yield.

During fermentative production of 1,5-pentanediamine, the endogenous lysE gene in a fermentation strain is replaced with a lysine decarboxylase gene cadA of Escherichia coli, thereby significantly increasing 1,5-pentanediamine yield.

The present invention provides use of the above-mentioned NADH-dependent amino acid dehydrogenase or coding genes thereof or a biological material containing the coding genes in the preparation of feed additives.

The present invention provides use of the above-mentioned NADH-dependent amino acid dehydrogenase or coding genes thereof or a biological material containing the coding genes in the preparation of medicines.

The NADH-dependent amino acid dehydrogenase according to the present invention can be used to replace a NADPH-dependent amino acid dehydrogenase of Corynebacterium glutamicum. This kind of particular amino acid dehydrogenases selected according to the present invention can directly utilize NADH as a cofactor or utilize both NADH and NADPH. In a strain for producing lysine or 1,5-pentanediamine, by directly expressing such NADH-dependent amino acid dehydrogenase, or replacing a corresponding NADPH-dependent amino acid dehydrogenase with the NADH-dependent amino acid dehydrogenase, cells can synthesize lysine by utilizing intracellular NADH as a cofactor, thereby reducing the demand of cells for NADPH and significantly increasing lysine yield or 1,5-pentanediamine yield.

### DETAILED DESCRIPTION OF THE INVENTION

The following examples are used for illustrating the present invention, but not to limit the scope of the present invention. Without departing from the spirit and essence of the present invention, modifications or alternations of methods, steps or conditions of the present invention all belong to the scope of the present invention.

Unless specifically indicated otherwise, chemical reagents that were used in the examples were all conventional reagents available in the market, and technological means that were used in the examples were conventional means well-known by persons skilled in the art.

### Example 1: Expression of NADH-Dependent Amino Acid Dehydrogenases and Verification of Catalytic Performance Thereof

Through numerous bioinformatics analysis and repeated comparative experiments, by expressing in Escherichia coli genes of amino acid dehydrogenases from different sources, detecting catalytic activity of the enzymes and detecting specificity on coenzymes NADH and NADPH, it was found that an aspartate dehydrogenase (with amino acid sequence of SEQ ID NO: 1) derived from Pseudomonas aeruginos, an aspartate semialdehyde dehydrogenase (with amino acid sequence of SEQ ID NO: 3) derived from Tistrella mobilis, a dihydrodipicolinic acid reductase (with amino acid sequence of SEQ ID NO: 5) derived from Mycobacterium tuberculosis, and a diaminopimelic acid dehydrogenase (with amino acid sequence of SEQ ID NO: 7) derived from Tepidanaerobacter acetatoxydans can catalyze reactions of the above four steps.

Based on the amino acid sequences of the above enzymes, corresponding codon optimized gene sequences, set forth in SEQ ID NOs: 2, 4, 6 and 8, were designed and genetically synthesized. Synthesized gene fragments were directly inserted into EcoRI and SalI double restriction sites of pET-28a to obtain plasmids designated as pET-adh, pET-asd, pET-dapB and pET-ddh, respectively. The plasmids were transformed into Escherichia coli BL21 (DE3) by chemical transformation, and recombinant bacteria were obtained by screening on an LB plate containing 50 mg/L kanamycin, designated as BL23/pET-adh, BL21/pET-asd, BL23/pET-dapB and BL23/pET-ddh, respectively.

The strains BL21 was cultured in an LB liquid medium containing 50 mg/L kanamycin until OD600 reached 0.6 (37° C., 150 rpm), 0.1 mM IPTG was added, and then the strains continued to be cultured for 12 h to induce protein expression (20° C., 150 rpm). The bacteria were centrifugally isolated, washed twice with 100 mL of 100 mM PBS buffer (pH 7.0), and finally resuspended in 5 mL of 100 mM PBS buffer (pH 7.0). The resuspension was broken up by ultrasonic waves, and centrifuged to obtain supernatant (12000 rpm, 30 min). The enzymes were isolated and purified by the Protein Purification kit HisTrap (GE), and was used for enzymatic activity assay.

The system for assaying aspartate dehydrogenase activity comprised: 100 mM Tris-HCl buffer (pH 8.2), 0.2 mM coenzyme NADH or NADPH, 4 mM oxaloacetic acid, 100 mM ammonium chloride and a proper amount of the enzyme. Reaction was carried out at 37° C., and the change of the absorbance of aspartate dehydrogenase at 340 nm was measured. The enzyme activity was defined as the amount (U) of the enzyme required for consuming 1 μM NAD(P)H per minute. Experimental results are shown in Table 1.

The system for assaying aspartate semialdehyde dehydrogenase activity comprised: 200 mM CHES buffer (pH 9.0), 50 mM KPi, 0.5 mM coenzyme NAD or NADP, 2 mM aspartate semialdehyde and a proper amount of the enzyme. Reaction was carried out at 25° C., and the change of absorbance of aspartate semialdehyde dehydrogenase at 340 nm was measured. The enzyme activity was defined as the amount (U) of the enzyme required for generating 1 μM NAD(P)H per minute. Experimental results are shown in Table 1.

The system for assaying dihydrodipicolinic acid reductase activity comprised: 100 mM HEPES buffer (pH 7.5), 0.2 mM coenzyme NADH or NADPH, 4 mM oxaloacetic acid, 1 mM pyruvic acid, 0.1 mM aspartate semialdehyde, 25 ug/mL dihydrodipicolinic acid synthetase and a proper amount of the enzyme. Reaction was carried out at 25° C., and the change of absorbance of dihydrodipicolinic acid reductase at 340 nm was measured. The enzyme activity was defined as the amount (U) of the enzyme required for consuming 1 μM NAD(P)H per minute. Experimental results are shown in Table 1.

The system for assaying diaminopimelic acid dehydrogenase activity comprised: 100 mM glycine-KOH buffer (pH 10.0), 0.5 mM coenzyme NAD or NADP, 5 mM diaminopimelic acid and a proper amount of the enzyme. Reaction was carried out at 30° C., and the change of absorbance of diaminopimelic acid dehydrogenase at 340 nm was measured. The enzyme activity was defined as the amount (U) of the enzyme required for generating 1 μM NAD(P)H per minute. Experimental results are shown in Table 1.

TABLE 1
Enzyme activity (unit: U/mg) of amino acid dehydrogenases from different sources

| | | Aspartate | | |
|---|---|---|---|---|
| | Aspartate | semialdehyde | Dihydrodipicolinic | Diaminopimelic |
| | dehydrogenase | dehydrogenase | acid reductase | acid dehydrogenase |
| | Pseudomonas | Tistrella | Mycobacterium | Tepidanaerobacter |
| Source | aeruginos | mobilis | tuberculosis | acetatoxydans |
| NADH | 34.5 | 6.7 | 5.6 | 12.6 |
| as substrate | | | | |
| NADPH | 37.2 | 7.7 | 1.8 | 1.1 |
| as substrate | | | | |

It can be seen from Table 1 that the four enzymes screened according to the present invention can all efficiently utilize NADH as a co-factor to catalyze target reactions, while the enzymes from Corynebacterium glutamicum cannot utilize NADH essentially.

### Example 2: Over-Expression of NADH-Dependent Amino Acid Dehydrogenases to Increase Lysine Yield of Corynebacterium glutamicum

In this example, the NADH-dependent amino acid dehydrogenases screened out according to the present invention were expressed, respectively, in the Corynebacterium glutamicum strain LC298 (Applied and environmental microbiology, 2011, 02912-10), and meanwhile corresponding NADPH-dependent amino acid dehydrogenases of Corynebacterium glutamicum were also expressed as the control. The effects of the enzymes specific for different coenzymes on promoting lysine synthesis were compared.

With the pET-adh as a template, using acagctatgacatgattacgaaggagatatacatatgctgaacattgtga-tgatcgg and tgcatgcctgcaggtcgactttagattgaaatggcatgggca as primers, the NADH-dependent aspartate dehydrogenase gene fragment was cloned. By utilizing a Gibson Assembly kit, the fragment was ligated into the EcoRI and XbaI sites of the plasmid pEC-K18mob2 (purchased from Addgene). The obtained plasmid was designated as pEC-adh_pa. The pEC-adh_pa was transformed by electroporation into the Corynebacterium glutamicum LC298 (electroporation conditions: voltage 2.5 kV, 2000 and 2 mm electroporation cup). A recombinant bacterium was obtained by screening on an LB plate containing 50 mg/L kanamycin, which was designated as LC/pEC-adh_pa, respectively. With the Corynebacterium glutamicum LC298 as a template, using acagctatgacatgattacgaaggagatatacatatgagttcagtttcgctgcagga and tgcatgcctgcaggtcgactttagttagcgt-aatgctccgctgc as primers, the aspartate aminotransferase gene fragment of Corynebacterium glutamicum itself was cloned. By utilizing a Gibson Assembly kit, the fragment was ligated into the EcoRI and XbaI sites of the plasmid pEC-K18mob2 (purchased from Addgene). The obtained plasmid was designated as pEC-aspC_cg. The pEC-aspC_cg was transformed by electroporation into the Corynebacterium glutamicum LC298 (electroporation conditions: voltage 2.5 kV, 200Ω and 2 mm electroporation cup). A recombinant bacterium was obtained by screening on an LB plate containing 50 mg/L kanamycin, which was designated as LC/pEC-aspC_cg, respectively.

With the pET-asd as a template, using acagctatgacatgattacgaaggagatatacatatgcgtatcgggattgt-tgga and tgcatgcctgcaggtcgactttacaccagtaactctgcgatttgc as primers, the NADH-dependent aspartate semialdehyde dehydrogenase gene fragment was cloned. By utilizing a Gibson Assembly kit, the fragment was ligated into the EcoRI and XbaI sites of the plasmid pEC-K18mob2 (purchased from Addgene). The obtained plasmid was designated as pEC-asd_tm. The pEC-asd_tm was transformed by electroporation into the Corynebacterium glutamicum LC298 (electroporation conditions: voltage 2.5 kV, 2000 and 2 mm electroporation cup). A recombinant bacterium was obtained by screening on an LB plate containing 50 mg/L kanamycin, which was designated as LC/pEC-asd_tm, respectively. Meanwhile, with the Corynebacterium glutamicum LC298 as a template, using acagctatgacatgattacgaaggagatatacatatgaccaccatcgcagttgtt and tgcatgcctgcaggtcgactttacttaaccagcagctcag as primers, the aspartate semialdehyde dehydrogenase gene fragment of Corynebacterium glutamicum itself was cloned. By utilizing a Gibson Assembly kit, the fragment was ligated into the EcoRI and XbaI sites of the plasmid pEC-K18mob2 (purchased from Addgene). The obtained plasmid was designated as pEC-asd_cg. The pEC-asd_cg was transformed by electroporation into the Corynebacterium glutamicum LC298 (electroporation conditions: voltage 2.5 kV, 20052 and 2 mm electroporation cup). A recombinant bacterium was obtained by screening on an LB plate containing 50 mg/L kanamycin, which was designated as LC/pEC-asd_cg, respectively.

With the pET-dapB as a template, using acagctatgacatgattacgaaggagatatacatatgcgggtaggcgtc-cttgg and tgcatgcctgcaggtcgactttacaaatttcagtgcagatcgagtagggg as primers, the NADH-dependent dihydrodipicolinic acid reductase gene fragment was cloned. By utilizing a Gibson Assembly kit, the fragment was ligated into the EcoRI and XbaI sites of the plasmid pEC-K18mob2 (purchased from Addgene). The obtained plasmid was designated as pEC-dapB_mt. The pEC-dapB_mt was transformed by electroporation into the Corynebacterium glutamicum LC298 (electroporation conditions: voltage 2.5 kV, 2000 and 2 mm electroporation cup). A recombinant bacterium was obtained by screening on an LB plate containing 50 mg/L kanamycin, which was designated as LC/pEC-dapB_mt. With the Corynebacterium glutamicum LC298 as a template, using acagctatgac-atgattacgaaggagatatacatatgggaatcaaggttggcgt and tgcatgcctgcaggtcgactttacaggcctaggtaatgctca as primers, the dihydrodipicolinic acid reductase gene fragment of Corynebacterium glutamicum itself was cloned. By utilizing a Gibson Assembly kit, the fragment was ligated into the EcoRI and XbaI sites of the plasmid pEC-K18mob2 (purchased from Addgene). The obtained plasmid was designated as pEC-pEC-dapB_cg. The pEC-dapB_cg was transformed by electroporation into the Corynebacterium glutamicum LC298 (electroporation conditions: voltage 2.5 kV, 200Ω and 2 mm electroporation cup). A recombinant bacterium was obtained by screening on an LB plate containing 50 mg/L kanamycin, which was designated as LC/pEC-dapB_cg.

With the pET-ddh as a template, using acagctatgacatgattacgaaggagatatacatatgccaaagaccaaa-gtgct and tgcatgcctgcaggtcgactttagaccagacgacaaattaattgttctaagtcg as primers, the NADH-dependent diaminopimelic acid dehydrogenase gene fragment was cloned. By utilizing a Gibson Assembly kit, the fragment was ligated into the EcoRI and XbaI sites of the plasmid pEC-K18mob2 (purchased from Addgene). The obtained plasmid was designated as pEC-ddh_ta. The pEC-ddh_ta was transformed by electroporation into the Corynebacterium glutamicum LC298 (electroporation conditions: voltage 2.5 kV, 200Ω and 2 mm electroporation cup). A recombinant bacterium was obtained by screening on an LB plate containing 50 mg/L kanamycin, which was designated as LC/pEC-ddh_ta, respectively. With the Corynebacterium glutamicum LC298 as a template, using acagctatgacatgattacgaaggagatatacatatgaccaacatccgcgtagcta and tgcatgcctgcaggtcgactttagacgtcgcgtgcgatca as primers, the diaminopimelic acid dehydrogenase gene fragment of Corynebacterium glutamicum itself was cloned. By utilizing a Gibson Assembly kit, the fragment was ligated into the EcoRI and XbaI sites of the plasmid pEC-K18mob2 (purchased from Addgene). The obtained plasmid was designated as pEC-ddh_cg. The pEC-ddh_cg was transformed by electroporation into the Corynebacterium glutamicum LC298 (electroporation conditions: voltage 2.5 kV, 20052 and 2 mm electroporation cup). A recombinant bacterium was obtained by screening on an LB plate containing 50 mg/L kanamycin, which was designated as LC/pEC-ddh_cg.

An empty plasmid pEC-K18mob2 was transformed by electroporation into the Corynebacterium glutamicum LC298 (electroporation conditions: voltage 2.5 kV, 2000 and 2 mm electroporation cup). A recombinant bacterium was obtained by screening on an LB plate containing 50 mg/L kanamycin, which was designated as LC/pEC-K18 and used as a control strain.

The above obtained derivative strains of the Corynebacterium glutamicum LC298 were respectively cultured in a fermentation medium for 72 h (30° C., 200 rpm), and then the lysine yield was assayed. The fermentation medium comprises (g/L): 80 g of glucose, 10 g of corn steep liquor, 4.5 g of urea, 45 g of ammonium sulfate, 0.5 g of potassium dihydrogen phosphate, 0.5 g of magnesium sulfate heptahydrate, 10 mg of ferrous sulfate heptahydrate, 10 mg of manganese sulfate tetrahydrate, 5 mg of β-alanine, 5 mg of nicotinic acid, 5 mg of thiamine hydrochloride, 0.3 mg of biotin, 30 g of calcium carbonate and 25 mg of kanamycin.

The lysine yield of the control strain LC/pEC-K18 was 14.01 g/L. Fermentation results of other strains are shown in Table 2, indicating that over-expression of NADH-dependent amino acid dehydrogenases can remarkably increase the lysine yield, and the effect is better that that of NADPH-dependent amino acid dehydrogenases from Corynebacterium glutamicum itself.

TABLE 2
Lysine yields (unit: g/L) of strains expressing amino
acid dehydrogenases specific for different coenzymes

| Aspartate dehydrogenase | | Aspartate semialdehyde dehydrogenase | |
|---|---|---|---|
| LC/pEC-adh_pa | LC/pEC-aspC_cg | LC/pEC-asd_tm | LC/pEC-asd_cg |
| 18.31 | 14.28 | 18.55 | 14.35 |

| Dihydrodipicolinic acid reductase | | Diaminopimelic acid dehydrogenase | |
|---|---|---|---|
| LC/pEC-dapB_mt | LC/pEC-dapB_cg | LC/pEC-ddh_ta | LC/pEC-ddh_cg |
| 17.32 | 14.15 | 19.98 | 18.75 |

### Example 3: Replacement of a NADPH-Dependent Amino Acid Dehydrogenase with a NADH-Dependent Amino Acid Dehydrogenase to Increase Lysine Yield

In addition to direct over-expression of a NADH-dependent amino acid dehydrogenase to increase lysine yield, lysine yield may be increased by direct replacement of a corresponding NADPH-dependent amino acid dehydrogenase in Corynebacterium glutamicum with a NADH-dependent amino acid dehydrogenase. In this example, in the Corynebacterium glutamicum strain ATCC21543, the NADPH-dependent amino acid dehydrogenases in cells were replaced with the NADH-dependent amino acid dehydrogenases screened out according to the present invention by homologous recombination, and the effects on improving lysine synthesis were observed.

With the pET-adh as a template, using gtacgcagttatgctgaacattgtgatgatcggatg and gctgtattcacttttagattgaaatggcatgggcatgattt as primers, the NADH-dependent aspartate dehydrogenase gene fragment adh was cloned. With the genome of ATCC21543 as a template, using acagctatgacatgattacggagttctttcttcagcgctgcg and tgttcagcataactgcgtacctccgcatgtg as primers, a gene fragment adh-up was cloned. With the genome of ATCC21543 as a template, using atttcaatctaaaagtgaatacagcggagacagc and tgcatgcctgcaggtcgactctcttcaacgattttcagcaaggc as primers, a gene fragment adh-down was cloned. By utilizing a Gibson Assembly kit, these three fragments were ligated into the EcoRI and XbaI sites of the pK18-mobsacB (purchased from Addgene), and the obtained plasmid was designated as pK18-adh. The pK18-adh was transformed by electroporation into the Corynebacterium glutamicum ATCC21543 (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM1:aspC. The main characteristic of this strain was that its endogenous aspartate transaminase was replaced with the NADH-dependent aspartate dehydrogenase.

With the pET-asd as a template, using tagttttacaatgcgtatcgggattgttggag and atggcgggtttttacaccagtaactctgcgatttgcac as primers, a NADH-dependent aspartate semialdehyde dehydrogenase gene fragment asd was cloned. With the genome of ATCC21543 as a template, using acagctatgacatgattacgagcccaatctttcacgggc and cgatacgcattgtaaaactactcctttaaaactttagcgtccg as primers, a gene fragment asd was cloned. With the genome of ATCC21543 as a template, using tactggtgtaaaaacccgccattaaaaactccg and tgcatgcctgcaggtcgactatttgtggtcattatctcggaaaaatgcg as primers, a gene fragment asd-down was cloned. By utilizing a Gibson Assembly kit, these three fragments were ligated into the EcoRI and XbaI sites of the pK18-mobsacB (purchased from Addgene), and the obtained plasmid was designated as pK18-asd. The pK18-asd was transformed by electroporation into the Corynebacterium glutamicum ATCC21543 (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM1:asd. The main characteristic of this strain was that its endogenous NADPH-dependent aspartate semialdehyde transaminase was replaced with the NADH-dependent aspartate semialdehyde dehydrogenase.

With the pET-dapB as a template, using aaggagcataatgcatgatgcaaacatccgc and tgaaatgagcctttacaaattattgagatcaagtacatctcgcatatcaaaaag as primers, a NADH-dependent dihydrodipicolinic acid reductase gene fragment dapB was cloned. With the genome of ATCC21543 as a template, using acagctatgacatgattacgctagatcgggctagatcgggctaa and catcatgcattatgctccttcattttcgtggggc as primers, a gene fragment dapB-up was cloned. With the genome of ATCC21543 as a template, using aataatttgtaaaggctcatttcagcagcgg and tgcatgcctgcaggtcgactttaaaagtccatgacatacgggcttgt as primers, a gene fragment dapB-down was cloned. By utilizing a Gibson Assembly kit, these three fragments were ligated into the EcoRI and XbaI sites of the pK18-mobsacB (purchased from Addgene), and the obtained plasmid was designated as pK18-dapB. The pK18-dapB was transformed by electroporation into the Corynebacterium glutamicum ATCC21543 (electroporation conditions: voltage 2.5 kV, 20092 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM1:dapB. The main characteristic of this strain was that its endogenous NADPH-dependent dihydrodipicolinic acid reductase was replaced with the NADH-dependent dihydrodipicolinic acid reductase.

With the pET-ddh as a template, using ttacaagaacatgccaaagaccaaagtgctg and tcgagctaaattagaccagacgacaaattaattgttctaagtcg as primers, a NADH-dependent diaminopimelic acid dehydrogenase gene fragment ddh was cloned. With the genome of ATCC21543 as a template, using acagctatgacatgattacgatcgctcaaggctgctgctg and tctttggcatgttcttgtaatcctccaaaattgt-ggtgg as primers, a gene fragment ddh-up was cloned. With the genome of ATCC21543 as a template, using tctggtctaatttagctcgaggggcaaggaa and tgcatgcctgcaggtcgactcttcccccgcaagacgatg as primers, a gene fragment ddh-down was cloned. By utilizing a Gibson Assembly kit, these three fragments were ligated into the EcoRI and XbaI sites of the pK18-mobsacB (purchased from Addgene), and the obtained plasmid was designated as pK18-ddh. The pK18-ddh was transformed by electroporation into the Corynebacterium glutamicum ATCC21543 (electroporation conditions: voltage 2.5 kV, 20052 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM1:ddh. The main characteristic of this strain was that its endogenous NADPH-dependent diaminopimelic acid dehydrogenase was replaced with the NADH-dependent diaminopimelic acid dehydrogenase.

The pK18-asd was transformed by electroporation into the Corynebacterium glutamicum AM1:aspC (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM2. The main characteristic of this strain was that its endogenous aspartate transaminase was replaced with the NADH-dependent aspartate dehydrogenase, and its endogenous NADPH-dependent aspartate semialdehyde transaminase was replaced with the NADH-dependent aspartate semialdehyde dehydrogenase.

The pK18-dapB was transformed by electroporation into the Corynebacterium glutomicum AM2 (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM3. The main characteristic of this strain was that its endogenous aspartate transaminase was replaced with the NADH-dependent aspartate dehydrogenase, its endogenous NADPH-dependent aspartate semialdehyde transaminase was replaced with the NADH-dependent aspartate semialdehyde dehydrogenase, and its endogenous NADPH-dependent dihydrodipicolinic acid reductase was replaced with the NADH-dependent dihydrodipicolinic acid reductase.

The pK18-ddh was transformed by electroporation into the Corynebacterium glutamicum AM3 (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain a correct recombinant strain, which was designated as AM4. The main characteristic of this strain was that its endogenous aspartate transaminase was replaced with the NADH-dependent aspartate dehydrogenase, its endogenous NADPH-dependent aspartate semialdehyde transaminase was replaced with the NADH-dependent aspartate semialdehyde dehydrogenase, its endogenous NADPH-dependent dihydrodipicolinic acid reductase was replaced with the NADH-dependent dihydrodipicolinic acid reductase, and its endogenous NADPH-dependent diaminopimelic acid dehydrogenase was replaced with the NADH-dependent diaminopimelic acid dehydrogenase.

The above obtained derivative strains of the Corynebacterium glutamicum ATCC21543 were respectively cultured in a fermentation medium for 72 h (30° C., 200 rpm), and then the lysine yield was assayed. The fermentation medium comprises (g/L): 80 g of glucose, 10 g of corn steep liquor, 4.5 g of urea, 45 g of ammonium sulfate, 0.5 g of potassium dihydrogen phosphate, 0.5 g of magnesium sulfate heptahydrate, 10 mg of ferrous sulfate heptahydrate, 10 mg of manganese sulfate tetrahydrate, 5 mg of β-alanine, 5 mg of nicotinic acid, 5 mg of thiamine hydrochloride, 0.3 mg of biotin, 30 g of calcium carbonate, 0.2 g of threonine and 0.2 g of leucine. Fermentation results of each of the strains are shown in Table 3, indicating that individual or combined replacement of the NADPH-dependent amino acid dehydrogenases in Corynebacterium glutamicum with the NADH-dependent amino acid dehydrogenases screened according to the present invention can remarkably increase the lysine yield.

TABLE 3
Lysine yields (unit: g/L) of recombinant strains
with amino acid dehydrogenase(s) replaced

| ATCC 21543 | AM1: aspC | AM1: asd | AM1: dapB | AM1: ddh | AM2 | AM3 | AM4 |
|---|---|---|---|---|---|---|---|
| 21.2 | 23.8 | 24.1 | 23.7 | 22.6 | 25.8 | 27.7 | 25.6 |

### Example 4: Increasing 1,5-Pentanediamine Yield by Using Corynebacterium glutamicum Containing NADH-Dependent Amino Acid Dehydrogenases

To express a lysine decarboxylase in the strains expressing the NADH-dependent enzymes can achieve the direct biosynthesis of 1,5-pentanediamine. Based on the strains obtained in Example 3, in this example, a lysine efflux gene lysE was replaced with a glutamic acid decarboxylase gene cadA from Escherichia coli by homologous recombination, thereby directly producing 1,5-pentanediamine from glucose and investigating the effects of NADH-dependent amino acid dehydrogenases on improving 1,5-pentanediamine synthesis.

With the Escherichia coli MG1655 as a template, using TTCGTGGTGTTGCCCGTGGCCCGGTTGGTTGGGCAGGAGTATATTGGGATCCatgAACGTTATTGCAATATTG AATC and catcaacatcagttaTTTTTTGCTTTCTTCTTTCAATAC as primers, a lysine decarboxylase gene fragment cadA was cloned. With the genome of ATCC21543 as a template, using acagctatgacatgattacgcgggcgaagaagtgaaaaacc and GCCACGGGCAACACCACGAATGCGCTACCTTAAC-CGAAAAGTTACTTTcgtgacctatggaagtacttaa as primers, a gene fragment cadA-up was cloned. With the genome of ATCC21543 as a template, using GCAAAAAAtaactgatgttgatgggttagttttcgc and tgcatgcctgcaggtcgactttcaacgcagcgcagcatta as primers, a gene fragment cadA-down was cloned. By utilizing a Gibson Assembly kit, these three fragments were ligated into the EcoRI and XbaI sites of the pK18-mobsacB (purchased from Addgene), and the obtained plasmid was designated as pK18-cadA. The pK18-cadA was respectively transformed by electroporation into the Corynebacterium glutamicum ATCC21543, AM1:aspC, AM1:asd, AM1:dapB, AM1:ddh, AM2, AM3 and AM4 (electroporation conditions: voltage 2.5 kV, 2000 and 2 mm electroporation cup). A recombinant bacterium was obtained by double screening. The primary recombinant bacterium was screened on an LB plate containing 25 mg/L kanamycin. This recombinant bacterium was further cultured overnight in a liquid LB medium and then was screened again on an LB plate containing 100 g/L sucrose to obtain correct recombinant strains, which were respectively designated as ATCC 21543-cadA, AM1:aspC-cadA, AM1:asd-cadA, AM1:dapB-cadA, AM1:ddh-cadA, AM2-cadA, AM3-cadA and AM4-cadA. The characteristic of these strains was that the endogenous lysE gene of each strain was replaced with the lysine decarboxylase gene cadA of Escherichia coli.

The above obtained strains of Corynebacterium glutamicum were respectively cultured in a fermentation medium for 72 h (30° C., 200 rpm), and then the lysine yield was assayed. The fermentation medium comprises (g/L): 80 g of glucose, 10 g of corn steep liquor, 4.5 g of urea, 45 g of ammonium sulfate, 0.5 g of potassium dihydrogen phosphate, 0.5 g of magnesium sulfate heptahydrate, 10 mg of ferrous sulfate heptahydrate, 10 mg of manganese sulfate tetrahydrate, 5 mg of β-alanine, 5 mg of nicotinic acid, 5 mg of thiamine hydrochloride, 0.3 mg of biotin, 30 g of calcium carbonate, 0.2 g of threonine and 0.2 g of leucine. Fermentation results of each of the strains are shown in Table 4, indicating that individual or combined replacements of the NADPH-dependent amino acid dehydrogenases in Corynebacterium glutamicum itself with the NADH-dependent amino acid dehydrogenases screened according to the present invention can remarkably increase the 1,5-pentanediamine yield.

TABLE 4
1,5-pentanediamine yields (unit: g/L) of recombinant
strains with amino acid dehydrogenase(s) replaced

| ATCC | | | |
|---|---|---|---|
| 21543-cadA | AM1:aspC-cadA | AM1:asd-cadA | AM1:dapB-cadA |
| 17.2 | 18.7 | 19.6 | 18.9 |
| AM1:ddh-cadA | AM2-cadA | AM3-cadA | AM4-cadA |
| 18.6 | 20.2 | 22.3 | 19.4 |

Although the present invention has been described with respect to general description and specific embodiments, it will be apparent to those skilled in the art that modifications or improvements can be made on the basis of the present invention. Therefore, such modifications or improvements made without departing from the spirit of the present invention all belong to the protection scope of the claims of the present invention.

1. NADH-dependent amino acid dehydrogenase, which is:
an aspartate dehydrogenase derived from Pseudomonas aeruginos;
an aspartate semialdehyde dehydrogenase derived from Tistrella mobilis;
a dihydrodipicolinic acid reductase derived from Mycobacterium tuberculosis; and/or
a diaminopimelic acid dehydrogenase derived from Tepidanaerobacter acetatoxydans;
the amino acid sequences thereof being set forth in SEQ ID NOs: 1, 3, 5 and 7, respectively, or being amino acid sequences obtained by substitution, deletion and/or addition of one or more amino acids in the amino acid sequences set forth in SEQ ID NOs: 1, 3, 5 and 7, respectively, without affecting bioactivity thereof.

2. A gene encoding the NADH-dependent amino acid dehydrogenase of claim 1, characterized in that its nucleotide sequence comprises the nucleotide sequence set forth in SEQ ID NOs: 2, 4, 6 and 8, respectively, or is a nucleotide sequence having a homology of 90% or more with the nucleotide sequence set forth in SEQ ID NOs: 2, 4, 6 and 8, respectively, obtained by substitution, deletion and/or addition of one or more bases, and coding a dehydrogenase of the same function.

3. A biological material comprising the gene of claim 2, characterized in that the biological material is a vector, a recombinant bacterium, a cell line or an expression cassette.

4. The biological material of claim 3, characterized in that the recombinant bacterium is a strain which is capable of fermentatively producing lysine or 1,5-pentanediamine and contains or over-expresses a NADH-dependent amino acid dehydrogenase which is
an aspartate dehydrogenase derived from Pseudomonas aeruginos;
an aspartate semialdehyde dehydrogenase derived from Tistrella mobilis;
a dihydrodipicolinic acid reductase derived from Mycobacterium tuberculosis; and/or
a diaminopimelic acid dehydrogenase derived from Tepidanaerobacter acetatoxydans;
the amino acid sequences thereof being set forth in SEO ID NOs: 1, 3, 5 and 7, respectively, or being amino acid sequences obtained by substitution, deletion and/or addition of one or more amino acids in the amino acid sequences set forth in SEO ID NOs: 1, 3, 5 and 7, respectively, without affecting bioactivity thereof.

5. The biological material of claim 4, characterized in that the recombinant bacterium is a strain which is capable of fermentatively producing lysine or 1,5-pentanediamine and
contains or over-expresses the aspartate dehydrogenase derived from Pseudomonas aeruginos and the aspartate semialdehyde dehydrogenase derived from Tistrella mobilis, or
contains or over-expresses the aspartate dehydrogenase derived from Pseudomonas aeruginos, the aspartate semialdehyde dehydrogenase derived from Tistrella mobilis and the dihydrodipicolinic acid reductase derived from Mycobacterium tuberculosis, or contains or over-expresses the aspartate dehydrogenase derived from Pseudomonas aeruginos, the aspartate semialdehyde dehydrogenase derived from Tistrella mobilis, the dihydrodipicolinic acid reductase derived from Mycobacterium tuberculosis and the diaminopimelic acid dehydrogenase derived from Tepidanaerobacter acetatoxydans.

6. The biological material of claim 3, characterized in that the recombinant bacterium is a strain which is capable of fermentatively producing lysine or 1,5-pentanediamine and in which NADPH-dependent amino acid dehydrogenase is replaced with a NADH-dependent amino acid dehydrogenase, which is
an aspartate dehydrogenase derived from Pseudomonas aeruginos;
an aspartate semialdehyde dehydrogenase derived from Tistrella mobilis;
a dihydrodipicolinic acid reductase derived from Mycobacterium tuberculosis; and/or
a diaminopimelic acid dehydrogenase derived from Tepidanaerobacter acetatoxydans;
the amino acid sequences thereof being set forth in SEO ID NOs: 1, 3, 5 and 7, respectively, or being amino acid sequences obtained by substitution, deletion and/or addition of one or more amino acids in the amino acid sequences set forth in SEO ID NOs: 1, 3, 5 and 7, respectively, without affecting bioactivity thereof.

7-10. (canceled)

11. The biological material of claim 4, characterized in that the recombinant bacterium is a strain which is capable of fermentatively producing lysine or 1,5-pentanediamide and in which NADPH-dependent amino acid dehydrogenase is replaced with a NADH-dependent amino acid dehydrogenase, which is
an aspartate dehydrogenase derived from Pseudomonas aeruginos;
an aspartate semialdehyde dehydrogenase derived from Tistrella mobilis;
a dihydrodipicolinic acid reductase derived from Mycobacterium tuberculosis; and/or
a diaminopimelic acid dehydrogenase derived from Tepidanaerobacter acetatoxydans;
the amino acid sequences thereof being set forth in SEQ ID NOs: 1, 3, 5 and 7, respectively, or being amino acid sequences obtained by substitution, deletion and/or addition of one or more amino acids in the amino acid sequences set forth in SEQ ID NOs: 1, 3, 5 and 7, respectively, without affecting bioactivity thereof.

12. The biological material of claim 5, characterized in that the recombinant bacterium is a strain which is capable of fermentatively producing lysine or 1,5-pentanediamine and in which NADPH-dependent amino acid dehydrogenase is replaced with a NADH-dependent amino acid dehydrogenase, which is
an aspartate dehydrogenase derived from Pseudomonas aeruginos;
an aspartate semialdehyde dehydrogenase derived from Tistrella mobilis;
a dihydrodipicolinic acid reductase derived from Mycobacterium tuberculosis; and/or
a diaminopimelic acid dehydrogenase derived from Tepidanaerobacter acetatoxydans;
the amino acid sequences thereof being set forth in SEQ ID NOs: 1, 3, 5 and 7, respectively, or being amino acid sequences obtained by substitution, deletion and/or addition of one or more amino acids in the amino acid sequences set forth in SEQ ID NOs: 1, 3, 5 and 7, respectively, without affecting bioactivity thereof.

13. A method for producing lysine or 1,5-pentanediamine, increasing lysine yield or 1,5-pentanediamine yield, or producing medicines or feed additives, comprising using the NADH-dependent amino acid dehydrogenase of claim 1 or a coding gene thereof.

14. The method of claim 13, characterized in that when 1,5-pentanediamine is fermentatively produced, an endogenous lysE gene in a fermentation strain is replaced with a lysine decarboxylase gene cadA of Escherichia coli.

15. A method for producing lysine or 1,5-pentanediamine, increasing lysine yield or 1,5-pentanediamine yield, or producing medicines or feed additives, comprising using the biological material of claim 3.

16. The method of claim 15, characterized in that when 1,5-pentanediamine is fermentatively produced, an endogenous lysE gene in a fermentation strain is replaced with a lysine decarboxylase gene cadA of Escherichia coli.


SEQUENCE LISTING (54 sequences)

SEQ ID NO: 1 - protein, 267 aa, Artificial Sequence
  an amino acid dehydrogenase
MLNIVMIGCGAIGAGVLELLENDPQLRVDAVIVPRDSETQVRHRLASLRRPPRVLSALPAGERPDLLVECAGHRAIEQHVLPALAQGIPCLVVSVGALSEPGLVERLEAAAQAGGSRIELLPGAIGAIDALSAARVGGLESVRYTGRKPASAWLGTPGETVCDLQRLEKARVIFDGSAREAARLYPKNANVAATLSLAGLGLDRTQVRLIADPESCENVHQVEASGAFGGFELTLRGKPLAANPKTSALTVYSVVRALGNHAHAISI

SEQ ID NO: 2 - DNA, 804 bp, Artificial Sequence
  a gene coding an amino acid dehydrogenase
atgctgaacattgtgatgatcggatgtggagcaattggtgcgggagttctggagcttcttgaaaacgaccctcagcttcgtgttgatgcagtaatcgtcccgcgcgactctgaaacacaggtacgccatcgtttggcatccctgcgtcgtccacctcgcgtattatcggccttgcctgcgggtgagcgcccggacctgttagtggagtgtgcaggacatcgcgctattgagcaacacgtcctgcctgcattggctcagggcatcccctgcctggtggtgtctgtgggtgcgttatcggaaccgggattggtagaacgtttagaagctgcggcccaagctggaggcagccgcattgaattactgcccggtgcaatcggagcaattgatgcactgagtgccgcacgtgttgggggattggaatccgtgcgttacactggtcgcaaacccgcttcggcatggcttggcacgcctggggaaacggtgtgcgacttacagcgtttggaaaaagcacgcgttatctttgacggcagcgcacgcgaagccgcccgcttatatcctaaaaatgccaacgtggcggcaaccctttctttagccggacttgggcttgatcgcacacaagtacgcttaattgcggaccccgagtcctgtgagaacgtacaccaggtggaggcttcaggcgcttttggcgggttcgagcttaccttgcgtggcaaaccattagcagcgaacccgaaaacgtcggctttaacagtgtactcagtggtccgtgctttaggaaatcatgcccatgccatttcaatctaa

SEQ ID NO: 3 - protein, 330 aa, Artificial Sequence
  an amino acid dehydrogenase
MRIGIVGATGAVGQETIQVLKDRGFPVTELHLFASERSAGKTTETAFGTITIEPFSVDAARGMDIVFLAVSGDFAKEYAPQIAAEGGAVVIDNSSAFRYDDAVPLVVPEINGRRALGQKLIANPNCTTAILLMALAPLHEAFGVKRAIVSTYQAASGAGAEGMTELEQGARQYLAGEPVTASKFAHPLAFNLIPHIDSFQDNGYTREEMKVLWETRKIMEAPEVLLSCTAVRVPTMRAHAEAVTIETRHPVTPAAAREVLAKAQGVTLADDPANKLYPMPLTASSKYDVEVGRIRESLVFGETGLDFFVCGDQLLKGAALNTVQIAELLV

SEQ ID NO: 4 - DNA, 993 bp, Artificial Sequence
  a gene coding an amino acid dehydrogenase
atgcgtatcgggattgttggagctacaggcgctgtgggccaggagactatccaagttctgaaggaccgtggcttccctgtcactgaactgcatttgtttgctagtgaacgttccgcaggaaagactacagaaacagccttcggaacaattacaatcgaaccattctctgtggacgcggctcgtggcatggacatcgttttcttagcagtcagcggagacttcgccaaagaatatgcgcctcaaattgctgcggaaggaggagcagtggtaattgataatagttccgcctttcgctacgacgacgcggtaccgctggtggtaccggaaattaatggacgccgtgcgttgggtcagaaactgatcgcgaaccccaattgtacaacggctatcctgcttatggcacttgcaccgttgcatgaagcatttggagtaaaacgcgctattgtttcgacctaccaagcagcttcaggcgcgggtgcggaaggaatgaccgaattggagcaaggggcccgccaatatcttgccggtgaacctgttacggcgtctaagttcgctcatccgctggcatttaatcttattccccacattgatagttttcaagataacggttacacacgcgaagagatgaaagtgttgtgggaaacacgtaagatcatggaagcacccgaagttcttttatcatgcaccgccgttcgtgtacccaccatgcgcgctcacgccgaggcggtaacgattgagacgcgccaccctgttactcctgccgctgcacgcgaggtgttagctaaggctcaaggcgtgacacttgcggacgatccagctaataaattgtacccaatgcccctgaccgcatctagcaagtacgacgtagaagtgggacgtattcgtgaatcgctggtgtttggcgaaacgggcctggacttcttcgtgtgcggggatcaactgctgaagggtgcggcattaaataccgtgcaaatcgcagagttactggtgtaa

SEQ ID NO: 5 - protein, 245 aa, Artificial Sequence
  an amino acid dehydrogenase
MRVGVLGAKGKVGATMVRAVAAADDLTLSAELDAGDPLSLLTDGNTEVVIDFTHPDVVMGNLEFLIDNGIHAVVGTTGFTAERFQQVESWLVAKPNTSVLIAPNFAIGAVLSMHFAKQAARFFDSAEVIELHHPHKADAPSGTAARTAKLIAEARKGLPPNPDATSTSLPGARGADVDGIPVHAVRLAGLVAHQEVLFGTEGETLTIRHDSLDRTSFVPGVLLAVRRIAERPGLTVGLEPLLDLH

SEQ ID NO: 6 - DNA, 738 bp, Artificial Sequence
  a gene coding an amino acid dehydrogenase
atgcgggtaggcgtccttggagccaaaggcaaggtcggaacgacaatggtgcgggcggtggccgccgccgacgacctgaccctatccgccgagctggatgccggcgatccgctgagcctgctaacggacggtaacaccgaggtcgtcatcgacttcacccacccggacgtggtgatgggcaatctggagttcctcatcgacaacggaattcacgccgtggtcggtaccacggggttcaccgccgagcggtttcaacaagtcgaatcgtggctcgtcgcaaaacccaacacatcggtgttgatagcgccaaacttcgcgatcggagcggtgctgtccatgcatttcgccaagcaggccgcacggtttttcgactcggccgaggtcattgagctgcatcatccgcacaaggctgacgcgccgtcaggcacggccgcgcgtaccgcgaagctgatcgccgaggcccgaaaaggcttgccgcccaatcccgatgccaccagtaccagcctgccgggcgcgcgtggtgccgacgtcgacggcataccggtgcacgcggtgcggctggccggactggtcgcccaccaggaagtgctgttcgggaccgagggggagactctgaccatccgccacgatagcctcgatcgcacatcgtttgtgcccggtgtgctgttggcggtgcgccgcatcgccgaacgccctggtctcaccgtaggtcttgagcccctactcgatctgcactga

SEQ ID NO: 7 - protein, 300 aa, Artificial Sequence
  an amino acid dehydrogenase
MQRVKVAIIGFGNVGKEVMGAVIESPDMEVAGIVEVPKKVECMKGKFHNFPVTSNVEKLDKVDIAILAVDSRCVPQIAPYYLERGINTVDAFDIHGDSIIRLREELTLVAKAHDAVAIISAGWDPGTNSVVRTIMQTIAPKGITYTNYGPGMSMGHTVAAKAVEGVADAVSLTIPEGNGIHKRLVYVKIKPDYDFKKIEEAIKNDSYFKHDTTIVYNVDDIENLIDMGHGVHIERKGVSGRTHNQRMEFIMQVTNPAATAQVMVSAARASLKQKPGAYTLAEIPPIDYLHGKKEEIILKL

SEQ ID NO: 8 - DNA, 906 bp, Artificial Sequence
  a gene coding an amino acid dehydrogenase
atgcagcgtgttaaggtggctattatcggttttggcaacgtaggaaaggaagtgatgggcgccgttatcgagtcccctgacatggaagttgcaggcattgtagaagtccctaaaaaagtggagtgtatgaaaggaaagtttcacaactttccggtaacgtccaatgtggagaaacttgacaaggtggatatcgctatcctcgctgtagattcacgttgtgtgcctcagatcgcaccttattatcttgaacgcggcatcaacacggtggatgcttttgacattcacggtgattcgattatccggctgcgggaggaactgacccttgttgccaaagcgcacgacgcagttgcgatcatctcggcgggttgggacccaggaactaattccgtagtgcgtactattatgcaaacgattgcgcccaagggaatcacttacactaactacggaccgggtatgagcatgggacacacggtagccgctaaagctgttgagggtgtggctgacgcggtctcactcaccattccggaaggcaatggcatccacaagcgcctcgtctacgtgaaaatcaagcccgactacgatttcaaaaagattgaggaagctattaagaacgactcttattttaagcatgatacgacgatcgtgtacaacgtggatgatatcgagaaccttatcgacatgggacacggagtacatattgagcgcaaaggcgttagcggacgtacccataaccaacgcatggagttcatcatgcaagtcactaaccccgctgccaccgcacaggtaatggtctcggcggcacgggcaagcctgaaacaaaagcccggagcgtacacgctcgcggaaatccctcctattgattaccttcacggcaaaaaggaagagatcattctcaaactgctgtaa

SEQ ID NO: 9 - DNA, 57 bp, Artificial Sequence
  primer
acagctatgacatgattacgaaggagatatacatatgctgaacattgtgatgatcgg

SEQ ID NO: 10 - DNA, 42 bp, Artificial Sequence
  primer
tgcatgcctgcaggtcgactttagattgaaatggcatgggca

SEQ ID NO: 11 - DNA, 57 bp, Artificial Sequence
  primer
acagctatgacatgattacgaaggagatatacatatgagttcagtttcgctgcagga

SEQ ID NO: 12 - DNA, 44 bp, Artificial Sequence
  primer
tgcatgcctgcaggtcgactttagttagcgtaatgctccgctgc

SEQ ID NO: 13 - DNA, 55 bp, Artificial Sequence
  primer
acagctatgacatgattacgaaggagatatacatatgcgtatcgggattgttgga

SEQ ID NO: 14 - DNA, 45 bp, Artificial Sequence
  primer
tgcatgcctgcaggtcgactttacaccagtaactctgcgatttgc

SEQ ID NO: 15 - DNA, 55 bp, Artificial Sequence
  primer
acagctatgacatgattacgaaggagatatacatatgaccaccatcgcagttgtt

SEQ ID NO: 16 - DNA, 40 bp, Artificial Sequence
  primer
tgcatgcctgcaggtcgactttacttaaccagcagctcag

SEQ ID NO: 17 - DNA, 54 bp, Artificial Sequence
  primer
acagctatgacatgattacgaaggagatatacatatgcgggtaggcgtccttgg

SEQ ID NO: 18 - DNA, 50 bp, Artificial Sequence
  primer
tgcatgcctgcaggtcgactttacaaatttcagtgcagatcgagtagggg

SEQ ID NO: 19 - DNA, 54 bp, Artificial Sequence
  primer
acagctatgacatgattacgaaggagatatacatatgggaatcaaggttggcgt

SEQ ID NO: 20 - DNA, 42 bp, Artificial Sequence
  primer
tgcatgcctgcaggtcgactttacaggcctaggtaatgctca

SEQ ID NO: 21 - DNA, 53 bp, Artificial Sequence
  primer
acagctatgacatgattacgaaggagatatacatatgccaaagaccaaagtgc

SEQ ID NO: 22 - DNA, 54 bp, Artificial Sequence
  primer
tgcatgcctgcaggtcgactttagaccagacgacaaattaattgttctaagtcg

SEQ ID NO: 23 - DNA, 56 bp, Artificial Sequence
  primer
acagctatgacatgattacgaaggagatatacatatgaccaacatccgcgtagcta

SEQ ID NO: 24 - DNA, 40 bp, Artificial Sequence
  primer
tgcatgcctgcaggtcgactttagacgtcgcgtgcgatca

SEQ ID NO: 25 - DNA, 36 bp, Artificial Sequence
  primer
gtacgcagttatgctgaacattgtgatgatcggatg

SEQ ID NO: 26 - DNA, 41 bp, Artificial Sequence
  primer
gctgtattcacttttagattgaaatggcatgggcatgattt

SEQ ID NO: 27 - DNA, 42 bp, Artificial Sequence
  primer
acagctatgacatgattacggagttctttcttcagcgctgcg

SEQ ID NO: 28 - DNA, 31 bp, Artificial Sequence
  primer
tgttcagcataactgcgtacctccgcatgtg

SEQ ID NO: 29 - DNA, 34 bp, Artificial Sequence
  primer
atttcaatctaaaagtgaatacagcggagacagc

SEQ ID NO: 30 - DNA, 44 bp, Artificial Sequence
  primer
tgcatgcctgcaggtcgactctcttcaacgattttcagcaaggc

SEQ ID NO: 31 - DNA, 32 bp, Artificial Sequence
  primer
tagttttacaatgcgtatcgggattgttggag

SEQ ID NO: 32 - DNA, 38 bp, Artificial Sequence
  primer
atggcgggtttttacaccagtaactctgcgatttgcac

SEQ ID NO: 33 - DNA, 39 bp, Artificial Sequence
  primer
acagctatgacatgattacgagcccaatctttcacgggc

SEQ ID NO: 34 - DNA, 43 bp, Artificial Sequence
  primer
cgatacgcattgtaaaactactcctttaaaactttagcgtccg

SEQ ID NO: 35 - DNA, 33 bp, Artificial Sequence
  primer
tactggtgtaaaaacccgccattaaaaactccg

SEQ ID NO: 36 - DNA, 49 bp, Artificial Sequence
  primer
tgcatgcctgcaggtcgactatttgtggtcattatctcggaaaaatgcg

SEQ ID NO: 37 - DNA, 31 bp, Artificial Sequence
  primer
aaggagcataatgcatgatgcaaacatccgc

SEQ ID NO: 38 - DNA, 54 bp, Artificial Sequence
  primer
tgaaatgagcctttacaaattattgagatcaagtacatctcgcatatcaaaaag

SEQ ID NO: 39 - DNA, 44 bp, Artificial Sequence
  primer
acagctatgacatgattacgctagatcgggctagatcgggctaa

SEQ ID NO: 40 - DNA, 34 bp, Artificial Sequence
  primer
catcatgcattatgctccttcattttcgtggggc

SEQ ID NO: 41 - DNA, 31 bp, Artificial Sequence
  primer
aataatttgtaaaggctcatttcagcagcgg

SEQ ID NO: 42 - DNA, 47 bp, Artificial Sequence
  primer
tgcatgcctgcaggtcgactttaaaagtccatgacatacgggcttgt

SEQ ID NO: 43 - DNA, 31 bp, Artificial Sequence
  primer
ttacaagaacatgccaaagaccaaagtgctg

SEQ ID NO: 44 - DNA, 44 bp, Artificial Sequence
  primer
tcgagctaaattagaccagacgacaaattaattgttctaagtcg

SEQ ID NO: 45 - DNA, 40 bp, Artificial Sequence
  primer
acagctatgacatgattacgatcgctcaaggctgctgctg

SEQ ID NO: 46 - DNA, 39 bp, Artificial Sequence
  primer
tctttggcatgttcttgtaatcctccaaaattgtggtgg

SEQ ID NO: 47 - DNA, 31 bp, Artificial Sequence
  primer
tctggtctaatttagctcgaggggcaaggaa

SEQ ID NO: 48 - DNA, 39 bp, Artificial Sequence
  primer
tgcatgcctgcaggtcgactcttcccccgcaagacgatg

SEQ ID NO: 49 - DNA, 77 bp, Artificial Sequence
  primer
ttcgtggtgttgcccgtggcccggttggttgggcaggagtatattgggatccatgaacgttattgcaatattgaatc

SEQ ID NO: 50 - DNA, 39 bp, Artificial Sequence
  primer
catcaacatcagttattttttgctttcttctttcaatac

SEQ ID NO: 51 - DNA, 41 bp, Artificial Sequence
  primer
acagctatgacatgattacgcgggcgaagaagtgaaaaacc

SEQ ID NO: 52 - DNA, 70 bp, Artificial Sequence
  primer
gccacgggcaacaccacgaatgcgctaccttaaccgaaaagttactttcgtgacctatggaagtacttaa

SEQ ID NO: 53 - DNA, 36 bp, Artificial Sequence
  primer
gcaaaaaataactgatgttgatgggttagttttcgc

SEQ ID NO: 54 - DNA, 40 bp, Artificial Sequence
  primer
tgcatgcctgcaggtcgactttcaacgcagcgcagcatta